(19) United States
(12) Patent Application Publication
(10) Pub. No.: US 2011/0142814 A1
(43) Pub. Date: Jun. 16, 2011

(54) Methods for Using Protein Kinase C-Theta Inhibitors in Adoptive Immunotherapy

(75) Inventors: Alexandra Zanin-Zhorov, Staten Island, NY (US); Michael L. Dustin, New York, NY (US)

(73) Assignee: New York University, New York, NY (US)

(21) Appl. No.: 12/968,690

(22) Filed: Dec. 15, 2010

Related U.S. Application Data
(60) Provisional application No. 61/286,871, filed on Dec. 16, 2009.

(51) Int. Cl. A61K 35/12; C12N 5/0783; A61P 37/06

(52) U.S. Cl. 424/93.71; 435/375

(57) ABSTRACT

The present invention provides methods for reducing tumor necrosis factor activation of regulatory T cells (Tregs), restoring the activity of defective Tregs, and enhancing the function of Tregs using Protein Kinase C theta (PKC-θ) inhibitors. The enhancement in Treg function is of use in facilitating adoptive immunotherapy in the treatment of immunological disorders.

### INTRODUCTION

This application claims benefit of priority to U.S. Provisional Application Ser. No. 61/286,871, filed Dec. 16, 2009, the content of which is incorporated herein by reference in its entirety.

This invention was made with government support under contract numbers PN2 EY016586 and R01 AI43542 awarded by the National Institutes of Health. The government has certain rights in the invention.

### BACKGROUND OF THE INVENTION

Protein kinase C-theta (PKC-θ) has been identified as a drug target for immunosuppression in transplantation and autoimmune diseases (Isakov, et al. (2002) Ann. Rev. Immunol. 20:761-794). WO 2004/043386 identifies PKC-θ as a target for treatment of transplant rejection and multiple sclerosis. PKC-θ also plays a role in inflammatory bowel disease (Banan, et al. (2005) J. Pharmacol. Exp. Therapeut. 313(3):962-982), asthma (WO 2005/062918), and lupus (Takeuchi, et al. (2005) Curr. Drug Targets Inflamm. Allergy 4:295-298). In addition, PKC-θ is highly expressed in gastrointestinal stromal tumors (Blay, et al. (2004) Clin. Can. Res. 10:12, Pt. 1) and has been suggested as a molecular target for treatment of gastrointestinal cancer (Wiedmann, et al. (2005) Curr. Cancer Drug Targets 5(3):171).

In this respect, PKC-θ inhibitors have been identified and suggested for use in the treatment of a variety of disease and disorders including cancer and autoimmune diseases (US 2010/0130486; US 2010/0120869; US 2008/0318929; US 2006/0217417; US 2005/0124640; US 20040166099; WO 2010/126967; WO 2008/051826; WO 2005/0066139; and Karihm, et al. (2007) J. Immunol. 178:B35).

### SUMMARY OF THE INVENTION

The present invention features methods for reducing tumor necrosis factor activation of regulatory T cells (Tregs), restoring the activity of a defective Treg, e.g., from a subject with rheumatoid arthritis, and enhancing the function of a Treg cell by contacting a Treg cell with a Protein Kinase C theta (PKC-θ) inhibitor. In some embodiments, the PKC-θ inhibitor is a protein-based, oligonucleotides-based or small organic molecule inhibitor.

A method for facilitating adoptive immunotherapy is also provided. This method involves contacting isolated Treg cells with a PKC-θ inhibitor and administering said Treg cells to a subject in need thereof to facilitate adoptive immunotherapy of the subject. In some embodiments, the claimed method prevents autoimmune colitis. In other embodiments, the subject has rheumatoid arthritis or graft rejection or graft-versus-host disease.

### DETAILED DESCRIPTION OF THE INVENTION

CD4⁺CD25⁺ regulatory T cells (Treg) suppress the function of CD4⁺ and CD8⁺ effector T cells (Teff) through a T cell receptor (TCR) engagement and cell contact-dependent mechanism (Sakaguchi, et al. (2008) Cell 133:775; Shevach, et al. (2006) Immunol. Rev. 212:60; Zheng & Rudensky (2007) Nat. Immunol. 8:457). Inflammatory signals delivered by cytokines like tumor necrosis factor (TNF)-α decrease Treg activity (Flores-Borja, et al. (2008) Eur. J. Immunol. 38:934; Valencia, et al. (2006) Blood 108:253), perhaps as a mechanism to reduce interference by Tregs in immune responses to pathogens. In rheumatoid arthritis, Tregs circulate in normal numbers, but they have decreased activity ex vivo (Valencia, et al. (2006) supra; Ehrenstein, et al. (2004) J. Exp. Med. 200:277). Besides the negative signals initiated by TNF-α, Treg also receive inhibitory signals via the TCR. Akt activation by the TCR can reduce Treg function and thus appears to be tightly regulated (Crellin, et al. (2007) Blood 109:2014). This suggests TCR signaling in Treg can negatively feed back to inhibit Treg-mediated suppression. Moreover, TCR signaling leads to formation of the immunological synapse within seconds of T cell activation. Thus, the differences in TCR signaling in Treg may emerge at the level of the immunological synapse (IS), a structured interface between T cells and antigen presenting cells where TCR signalosomes are assembled (Dustin, et al. (2006) Curr. Opin. Immunol. 18:512). Whereas Treg can form stable contacts with APCs with functional consequences both in vitro and in vivo (Tang, et al. (2006) Nat. Immunol. 7:83; Tran, et al. (2009) J. Immunol. 182:2929; Wing, et al. (2008) Science 322:271), signaling events in the Treg IS have not been investigated.

Protein kinase C-theta (PKC-θ) recruitment to the immunological synapse is required in effector cells to recruit Carma-1 in the pathway to NF-κB activation. It has now been found that PKC-θ is sequestered away from the Treg immunological synapse. Furthermore, PKC-θ inhibition or suppression by RNAi enhances Treg function, demonstrating PKC-θ-mediated negative feedback. Treg integrate TCR and TNF signals through control of PKC-θ location such that inhibition of PKC-θ protects Treg from inactivation by TNF, restores activity of defective Treg from rheumatoid arthritis patients, and enhances protection of mice from Inflammatory Colitis. Treg, freed of PKC-θ-mediated negative feedback, can function in the presence of TNF and thus have therapeutic potential in control of inflammatory diseases. Accordingly, the present invention features methods for using PKC-θ inhibitors to reduce or protect Treg from TNF inactivation, restore activity of defective Treg, enhance Treg function, and in adoptive immunotherapy applications.

As is known in the art, PKC-θ (also known as PKC-theta, PKCT, PRKCT, nPKC-theta and PRKCQ) is a calcium-independent, diacylglycerol (DAG)-dependent serine/threonine protein kinase that is expressed in testicular interstitial cells, capillary endothelial cells, skeletal muscle cells, neural cells and various hematopoietic cells, including megakaryocyte and erythroblast progenitor lines, platelets, mast cells, thymocytes, T-lymphomas, and mature T-cells. This isozyme has been shown to be specifically responsible for antigen driven activation events in peripheral T cells. PKC-θ is not required for the development of T cells in the thymus, as PKC-theta knock-out mice develop normal numbers of peripheral T cells (Sun, et al. (2000) Nature 404:402-7). However, when these mice are challenged with an antigen, they fail to make a T cell response. Originally identified from a human peripheral blood lymphocyte-derived cDNA library (Baier, et al. (1993) J. Biol. Chem. 268:4997-5004), the polypeptide and nucleic acid sequence encoding human PKC-θ are known in the art and available under GENBANK Accession Nos. NP_{—}006248 and NM_{—}006257, respectively.

In accordance with the instant methods, PKC-θ is inhibited to, e.g., protect Treg from TNF inactivation, restore activity of defective Treg, and enhance Treg function. In particular embodiments, the PKC-θ inhibitor is selective for PKC-θ and exhibits no or significantly less activity against other enzymes including PKC isozymes such as classical or conventional PKCs (subtypes alpha, beta and gamma), novel PKCs (delta, epsilon, eta, and mu) and atypical PKCs (zeta, tau/lambda). In certain embodiments, the PKC-θ inhibitor has an IC₅₀ of equal to or less than 10 nM, 5 nM, 1 nM or 100 pM. In particular embodiments, the PKC-θ inhibitor has an IC_{H} in the range of 100 pM to 1.

A variety of PKC-θ inhibitors can be used in accordance with the present invention and, for the purposes herein are classified into three groups, small organic molecules, protein-based, and oligonucleotide-based inhibitors. Examples of suitable small organic PKC-θ inhibitors include, but are not limited to, those exemplified herein as well as 2,4-pyrimidinediamine compounds and prodrugs thereof (see US 2010/0130486); 2,4-diamino-5-nitropyrimidines (see Cywin, et al. (2007) Bioorg. Med. Chem. Lett. 17:225-230); Rottlerin (Springael, et al. (2007) Biochem. Pharmacol. 73:515-25); Sotrastaurin (AEB071, Skvara, et al. (2008) J. Clin. Invest. 118:3151-3159); Compound A (see Chaudhary, et al. (2009) J. Immunol. 182:93.21); purines (see WO 2008/051826); pyrimidine derivatives (see US 2008/0318929); pyridine derivatives (see US 2006/0217417); 4-(substituted phenoxy)-anilines, 4-(halogenated phenoxy)-anilines, 4-(substituted phenylthio)-anilines, 4-[substituted (phenylsulfinyl and phenylsulfonyl)]-anilines, N-acylated 4-(substituted phenylthio)anilines, 4-(tert-butylphenylthio)acetanilide; and N-acylated 4-[substituted (phenylsulfinyl and phenylsulfonyl)]-anilines (see US 2010/0120869); and 2-(amino-substituted)-4-aryl pyramidines (WO 2005/0066139).

For the purposes of the present invention, a “protein-based” or “peptide-based” PKC-θ inhibitor is an inhibitor composed of two or more amino acid residues covalently attached by peptide bonds, which may be further modified to include organic and/or inorganic groups. Protein-based PKC-θ inhibitors can be PKC-θ binding proteins that bind to PKC-θ but do not activate the enzyme. Specific examples of suitable protein-based PKC-θ inhibitors include, but are not limited to, PKC-theta inhibitory factor (PIF) peptide (Tas, et al. (2005) Arthritis Res. Ther. 7 (Suppl. 1):P1), pseudosubstrate inhibitors such as LHQRRGAIKQAKVHHVKC-NH₂ (SEQ ID NO:3; EMD Chemicals), and RACK peptide (Receptor for Activated C Kinase; Nagy, et al. (2009) Blood 114:489-91). As described herein, protein-based conjugates are also of use in the instant methods. Moreover, an antibody or antibody fragment that specifically binds to PKC-θ protein (e.g., an antibody that disrupts PKC-θ's catalytic activity or an antibody that disrupts the ability of upstream activators to activate PKC-θ) is also included within the scope of a protein-based PKC-θ inhibitor of the invention. Such antibodies and fragments are routinely produced in the art and can be assayed for inhibitory activity as described herein. Alternatively, antibodies to human PKC-θ may be obtained from Invitrogen, Carlsbad, Calif.; BD Biosciences Pharmigen, San Diego, Calif.; Novus Biologicals, Littleton, Colo.; or Epitomics, Inc., Burlingame, Calif.

“Oligonucleotide-based” inhibitors include molecules composed of two or more nucleotides (RNA or DNA) and/or peptide-nucleic acids that inhibit the expression and/or activity. Typically, oligonucleotides-based inhibitors decrease the level of expression of an endogenous PKC-θ gene (e.g., by decreasing transcription of the PKC-θ gene). In particular embodiments, oligonucleotides-based inhibitors include interfering RNA (RNAi), antisense oligonucleotides (ODNs), ribozymes and DNAzymes as sequence-specific inhibitors of gene expression.

Antisense oligonucleotides can be complementary to the entire coding region of mRNA, but more preferably is an oligonucleotide which is antisense to only a portion of the coding or noncoding region of mRNA. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation. Non-limiting examples of antisense nucleic acids are disclosed in U.S. Pat. No. 6,190,869, which is incorporated herein by reference in its entirety.

RNA-interfering PKC-θ inhibitors are small nucleic acid molecules that downregulate, inhibit, or reduce the expression of PKC-θ. Non-limiting examples of such nucleic acid molecules include short interfering nucleic acid (“siNA”), short interfering RNA (“siRNA”), double stranded RNA (“dsRNA”), micro-RNA (“miRNA”), and short hairpin RNA (“shRNA”). Techniques for making these nucleic acid molecules are disclosed, for example, in U.S. Pat. Nos. 5,514,567; 5,561,222; 6,506,559; 7,022,828; 7,078,196; 7,176,304; 7,282,564; and 7,294,504; which are incorporated herein by reference in their entirety. Examples of suitable siRNA target sequences are disclosed herein as SEQ ID NOs:1 and 2. Alternatively, useful PKC-θ RNAis may be those available from Invitrogen, Carlsbad, Calif.

Conjugates and mixtures of the above-referenced inhibitors are also within the scope of the present invention. For example, particular embodiments include the use of bisubstrate inhibitors targeting both the ATP and substrate binding site of the catalytic domain of PKC-θ (Broom (1989). J. Med. Chem. 32:2-7).

The inhibitors described herein can be used as is or administered in combination with a physiologically or pharmaceutically acceptable carrier, excipient, or stabilizer. The term “pharmaceutically acceptable” means a non-toxic material that does not interfere with the effectiveness of the biological activity of the active ingredients. The term “pharmaceutically-acceptable carrier” means one or more compatible solid or liquid fillers, diluents or encapsulating substances which are suitable for administration to a human or other vertebrate animal. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. In the preferred embodiment, administration is by injection. Typical formulations for injection include a carrier such as sterile saline or a phosphate buffered saline. Viscosity modifying agents and preservatives are also frequently added.

Optional pharmaceutically acceptable excipients especially for enteral, topical and mucosal administration, include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Binders are used to impart cohesive qualities to a solid dosage formulation, and thus ensure that a tablet or bead or granule remains intact after the formation of the dosage forms. Lubricants are used to facilitate tablet manufacture. Disintegrants are used to facilitate dosage form disintegration or “breakup” after administration, and generally include, but are not limited to, starch, sodium starch glycolate, alginine, gums or cross linked polymers, such as cross-linked PVP. Stabilizers are used to inhibit or retard decomposition reactions which include, by way of example, oxidative reactions. Surfactants may be anionic, cationic, amphoteric or nonionic surface active agents. If desired, the compositions may also contain minor amount of nontoxic auxiliary substances such as wetting or emulsifying agents, dyes, pH buffering agents, or preservatives.

As described herein, inhibition of PKC-θ finds application in in vivo, ex vivo and in vitro methods of reducing or protecting Treg from TNF inactivation, restoring activity of defective Treg, and enhancing Treg function. In accordance with these methods, Treg cells are contacted with one or more PKC-θ inhibitors, as described herein, to free the Treg from PKC-θ-mediated negative feedback. As used herein, the term “regulatory T cell” or “Treg” includes T cells that produce low levels of IL-2, IL-4, IL-5, and IL-12. Regulatory T cells produce TNFα, TGFβ, IFN-γ, and IL-10, albeit at lower levels than effector T cells. Although TGFβ is the predominant cytokine produced by regulatory T cells, the cytokine is produced at levels less than or equal to that produced by Th1 or Th2 cells, e.g., an order of magnitude less than in Th1 or Th2 cells. Regulatory T cells can be found in the CD4⁺CD25⁺ population of cells (see, e.g., Waldmann & Cobbold (2001) Immunity 14:399). Regulatory T cells actively suppress the proliferation and cytokine production of Th1, Th2, or naïve T cells which have been stimulated in culture with an activating signal (e.g., antigen and antigen presenting cells or with a signal that mimics antigen in the context of MHC, e.g., anti-CD3 antibody, plus anti-CD28 antibody). Accordingly, use of a PKC-θ inhibitor can enhance the function of a Treg by increasing its ability to suppress the proliferation and cytokine production of Th1, Th2, or naïve T cells as compared Treg cells not contacted with the inhibitor. For ex vivo and in vitro aspects of this invention, the Treg cells can be isolated and optionally purified from peripheral blood leukocytes, bone marrow, lymph node tissue, spleen tissue, and tumors. A “defective” Treg is a Treg with low FoxP3 expression and reduced or deficient effector functions. See, e.g., Gousteri & von Herrath (2006) Blood 108:3-4. In this respect, restoration of the activity of a defective regulatory T cell means that FoxP3 expression and effector functions are returned to normal levels.

Methods of protecting Treg from TNF inactivation, restoring activity of defective Treg, and enhancing Treg function find application in facilitating adoptive immunotherapy. Adoptive immunotherapy refers to a therapeutic approach in which immune cells are administered to a subject, with the aim that the cells mediate either directly or indirectly the treatment or prevention of diseases and disorders characterized by undesirable activation, overactivation or inappropriate activation of the immune system, such as occurs during immunological disorders such as allergic responses, autoimmune diseases and disorders, graft rejection and graft-versus-host-disease. In accordance with the present invention, diseases associated with activation, overactivation or inappropriate activation of the immune system are treated or prevented by adoptive immunotherapy using regulatory T cells (Tregs) treated with the PKC-θ inhibitors and methods disclosed herein.

In accordance with the methods described herein, T cells are obtained from the subject to be treated, and a Treg enriched cell population is obtained by negative and/or positive selection, e.g., using cell sorting via negative magnetic immunoadherence, which utilizes a cocktail of monoclonal antibodies directed to cell surface markers present on the cells negatively selected. To maximize efficacy, the subpopulation is enriched to at least 90%, preferably at least 95%, and more preferably at least 98% Treg cells, preferably CD4⁺CD25⁺ Treg cells. Positive selection may be combined with negative selection against cells having surface makers specific to non-Treg cell types, such as depletion of CD8, CD11b, CD16, CD19, CD36 and CD56-bearing cells.

In some embodiments, the Treg cells are activated in a polyclonal or antigen-specific manner ex vivo, expanded, and administered to the subject to be treated according to established methods (see June & Blazar (2006) Sem. Immunol. 18:78; Hippen, et al. (2008) Blood 112:2847). In another embodiment, a population of T cells not enriched for Treg cells is activated and expanded, and the Treg cells are selected from the expanded T cell population using appropriate positive and/or negative selection.

Following or during isolation, activation and/or expansion of the T cells, Tregs are contacted with a PKC-θ inhibitor and administered to the subject in amounts effective to induce an immune response. The immune response induced in the animal by administering the compositions may include cellular immune responses mediated by CD4⁺ T cell responses and humoral immune responses, mediated primarily by B cells that produce antibodies following activation by CD4⁺ T cells. A variety of techniques which are well known in the art may be used for analyzing the type of immune responses induced by the compositions and methods disclosed herein (Coligan, et al. (1994) Current Protocols in Immunology, John Wiley & Sons Inc.).

In some embodiments of the instant methods, the Treg cells are assayed to ensure that the kinase activity of PKC-θ is inhibited. Assays for measuring PKC-θ activity are known in the art and include, but are not limited to, competitive Fluorescent Polarization Assays and antibody-based assays measuring phosphorylation of substrates.

Adoptive immunotherapy using Treg cells can be used for prophylactic and therapeutic applications in immunological disorders. In prophylactic applications, Treg cells are administered in amounts effective to eliminate or reduce the risk or delay the onset of conditions associated with undesirable activation, over-activation or inappropriate or aberrant activation of an immune response, including physiological, biochemical, histologic and/or behavioral symptoms of the disorder, its complications and intermediate pathological phenotypes presenting during development of the disease or disorder. In therapeutic applications, the compositions and methods disclosed herein are administered to a patient suspected of, or already suffering from such a condition associated with undesirable activation, over-activation or inappropriate or aberrant activation of an immune response to treat, at least partially, the symptoms of the disease (physiological, biochemical, histologic and/or behavioral), including its complications and intermediate pathological phenotypes in development of the disease or disorder. An amount adequate to accomplish therapeutic or prophylactic treatment is defined as a therapeutically- or prophylactically-effective amount.

In certain embodiments, the immunological disorder being treated is selected from psoriasis, rheumatoid arthritis, multiple sclerosis, type I diabetes, inflammatory bowel disease, Guillain-Barre syndrome, Crohn's disease, ulcerative colitis, graft versus host disease (and other forms of organ or bone marrow transplant rejection), and systemic lupus erythematosus. In one embodiment, the immunological disorder being treated is rheumatoid arthritis. In another embodiment, the immunological disorder being treated is autoimmune colitis. In a further embodiment, the immunological disorder being treated is graft rejection or graft-versus-host disease. Subjects, individuals, or patient benefiting from treatment include, but not limited to, murines, simians, humans, mammalian farm animals, mammalian sport animals, and mammalian pets.

With respect to allograft rejection or graft versus host disease, in a particular embodiment, adoptive immunotherapy with Treg cells is initiated prior to transplantation of the allograft. In certain embodiments, the Treg cells can be administered to the subject for a day, three days, a week, two weeks or a month prior to transplantation. In other embodiments, the Treg cells are administered for a week, two weeks, three weeks, one month, two months, three months or six months following a transplantation. In a preferred embodiment, Treg cells are administered both before and after transplantation is carried out.

The outcome of the therapeutic and prophylactic methods disclosed herein is to at least produce in a patient a healthful benefit, which includes, but is not limited to, prolonging the lifespan of a patient, delaying the onset of one or more symptoms of the disorder, and/or alleviating a symptom of the disorder after onset of a symptom of the disorder. For example, in the context of allograft rejection, the therapeutic and prophylactic methods can result in prolonging the lifespan of an allograft recipient, prolonging the duration of allograft tolerance prior to rejection, and/or alleviating a symptom associated with allograft rejection.

The invention is described in greater detail by the following non-limiting examples.

### Example 1

### Materials and Methods

Cell Purification. CD4⁺CD25⁺ and CD4⁺CD25⁻ T cells were purified from the peripheral blood of healthy human donors between ages of 16 and 75 years (Zanin-Zhorov, et al. (2006) J. Clin. Invest. 116:2022) (New York Blood Center, NY, N.Y.) or from 25 patients with Rheumatoid arthritis in different stages (accordingly to disease activity score (DAS); Table 1) according to established methods (Prevoo, et al. (1995) Arthritis Rheum. 38:44).

TABLE 1

| | | | | | DAS | |
|---|---|---|---|---|---|---|
| Sample | Age | Gender | Ethnicity | DMARD | Score | ESR |
| 100486 | 38 | M | Asian | MTX | 0.84 | 3 |
| 100489 | 42 | F | Hispanic | MTX | 1.75 | 11 |
| 100480 | 59 | M | NHW | MTX | | 15 |
| 100485 | 51 | M | Hispanic | None | 3.61 | 12 |
| 100479 | 76 | F | NHW | MTX | 3.7 | 20 |
| 100469 | 59 | F | Hispanic | None | 3.77 | 73 |
| 100470 | 46 | F | Hispanic | MTX | 3.81 | 12 |
| 100471 | 38 | M | Asian | None | 3.9 | 4 |
| 100487 | 24 | F | NHW | None | 4.26 | 44 |
| 100464 | 44 | F | Hispanic | None | 4.36 | 19 |
| 100488 | 61 | F | Hispanic | None | 4.48 | 52 |
| 100442 | 62 | F | Hispanic | MTX | 4.49 | 27 |
| 100481 | 59 | F | Hispanic | MTX | 4.6 | 38 |
| 100441 | 61 | M | Hispanic | MTX | 4.88 | 14 |
| 100477 | 46 | F | Asian | MTX | 4.89 | 18 |
| 100463 | 52 | F | AA | MTX | 5.59 | 93 |
| 100476 | 48 | F | NHW | MTX | 5.98 | 8 |
| 100483 | 48 | F | NHW | MTX | 5.99 | 12 |
| 100484 | 52 | F | AA | MTX | 6.08 | 30 |
| 100473 | 50 | F | AA | MTX | 6.53 | 14 |
| 100491 | 50 | F | Hispanic | MTX | 6.64 | 12 |
| 100492 | 76 | F | AA | MTX | 6.64 | 32 |
| 100482 | 69 | F | NHW | None | 7.35 | |
| 100493 | 58 | F | Hispanic | MTX | 7.45 | 62 |
| 100494 | 70 | F | Hispanic | MTX | 8.17 | 60 |

M, Male;
F, Female;
NHW, Non-Hispanic White;
AA, African American;
DMARD, Disease Modifying Anti-Rheumatic Drug;
MTX, Methodexate;
DAS, Disease Activity Score;
ESR, Erythrocyte Sedimentation Rate.

Briefly, the whole blood was incubated (20 minutes, 22° C.) with ROSETTESEP human CD4⁺ T cell enrichment cocktail (StemCell Technologies, Vancouver, BC, Canada). The remaining unsedimented cells were loaded onto FICOLL-PAQUE Plus (Amersham Bioscience, Piscataway, N.J.), isolated by density centrifugation, and washed with phosphate-buffered saline (PBS). In the second round of purification, CD4+ T cells were separated into CD25⁻ and CD25⁺ populations with magnetically coupled monoclonal antibody against human CD25 (Miltenyi Biotec, Bergisch Gladbach, Germany). The purified cells were cultured in RPMI medium containing antibiotics and 10% heat-inactivated fetal bovine serum (FBS). Human antigen-presenting cells (APCs) were obtained by depleting T cells from peripheral blood mononuclear cells (PBMCs) with CD3⁺ depletion kit (StemCell Technologies, Vancouver, BC, Canada) and treated with mitomycin C 0.2 mg/ml for 30 minutes, washed and resuspended in complete medium. CD4⁺CD25^{hi} T cells were separated from total CD4⁺ T cells according to their CD25 expression by means of high-speed FACS sorting. CD4⁺ T cells were incubated for 30 minutes with anti-CD25 PE-labeled antibodies (Miltenyi Biotec). Sorting was performed by using a MOFLO cytometer (BeckmanCoulter, Brea, Calif.). Umbilical cord blood (UCB) CD25⁺ and CD25⁻CD4⁺ T cells were isolated from frozen UCB units (National Placental Blood Program, New York Blood Center) by positive selection using directly conjugated anti-CD25 magnetic microbeads. Cells were cultured with anti-CD3/CD28 monoclonal antibody-coated DYNABEADS for 18 to 21 days and split every 2 to 3 days. Recombinant IL-2 (300 IU/ml; Chiron, Emeryville, Calif.) was added on day 3 and maintained for culture duration.

Planar Lipid Bilayers. Planar lipid bilayers were prepared in parallel-plate flow cells according to established methods (Dustin, et al. (2007) Curr. Protoc. Immunol. 7:13; Grakoui, et al. (1999) Science 285:221; Kaizuka, et al. (2007) Proc. Natl. Acad. Sci. USA 104:20296). Liposomes that contained biotin-CAP-phosphatidylethanolamine (Avanti Polars Lipids, Alabaster, Ala.) were mixed with liposomes containing CY5-ICAM-1-GPI and placed on an acid Piranha solution cleaned 40 mm round glass coverslip to form the planar bilayers with final density 0.01 mol % biotin and ICAM-1 250 molecules/μm². After blocking streptavidin (4 μg/ml) and ALEXA FLUOR 546-labeled monobiotinylated anti-CD3ε monoclonal antibody (5 μg/ml, OKT3, eBioscience, San Diego, Calif.) were reacted sequentially with the biotinylated lipid bilayers. The flow cell containing the bilayers was warmed up to 37° C., cells were injected in 500 μl of HEPES-buffered saline containing 1% human serum albumin, and images were collected for 30 minutes on a custom automated OLYMPUS IX-70 inverted fluorescence microscope.

Microscopy. All TIRF imaging was performed on the custom automated OLYMPUS IX-70 inverted fluorescence microscope using the 60×/1.45 N.A. TIRF objective from OLYMPUS. TIRF illumination was set up and aligned according to the manufacturer's instructions (Varma, et al. (2006) Immunity 25:117). Briefly, cells interacted with the bilayers for 6 minutes at 37° C., fixed with 2% paraformaldehyde (PFA), permeabilized with 0.05% TRITON-X 100, and blocked. Cells were incubated with rabbit polyclonal antibodies to phospho-Src tyr 416 (Cell Signaling Technologies, Beverly, Mass.), phospho-ZAP-70 tyr 319 (sc-12946-R, Santa Cruz Biotech, Santa Cruz, Calif.), PKC-θ (sc-212; Santa Cruz Biotech) or Carma-1 (Card 10, C-12, Santa Cruz Biotech) for 20 minutes, and then incubated with fluorescently tagged goat anti-rabbit Fab2 (Invitrogen, Carlsbad, Calif.). Controls included the use of nonimmune species-matched IgG. Measurement of signaling was done by determining fluorescence intensity on the entire cell-bilayer contact area as detected in the IRM channel. Average fluorescence intensity was measured using IP-Lab software (Biovision, Exton, Pa.) and background fluorescence was subtracted from average intensity. Confocal microscopy was carried out on a ZEISS LSM 510 Meta imaging system (63×1.4 NA; ZEISS, Jena, Germany) using appropriate factory-set filters and dichroics for different fluorophores. Fluorophore saturation was cautiously avoided during acquisition. Acquisition settings were maintained constant throughout the experiment. Images were acquired using LSM (ZEISS) software and were analyzed and reconstructed in 3D using Imaris software (Bitplane, Saint Paul, Minn.). The images were further processed using PHOTOSHOP under identical contrast settings.

In Vitro Suppression Assays. CD4⁺CD25⁺ T cells were treated or not, washed, and added at different ratios (1:9, 1:3, 1:1 or 5×10⁴:5×10⁵, 1.25×10⁵:5×10⁵, 5×10⁵:5×10⁵, respectively) to CD4⁺CD25⁻ T cells at final concentration 2×10⁶/ml (cytokine secretion) or 2×10⁵/ml (proliferation). The cells were co-cultured on anti-CD3 monoclonal antibody (5 μg/ml) pre-coated 24-well plates for 24-48 hours (cytokine secretion), or 96 hours (proliferation). Human TNF-α (210-TA) and neutralizing antibodies against TGF-β RII (AF-241-NA) were purchased from R&D Systems Inc. (Minneapolis, Minn.) and added to co-cultures where indicated. Cytokine secretion was determined by ELISA (Zanin-Zhorov, et al. (2006) supra), using Human IFN-γ CYTOSET and Human IL-10 CYTOSET (Biosource; Camarillo, Calif.) and IL-17 and IL-4 (Invitrogen). Proliferation was assessed by ALAMAR BLUE assay (Invitrogen) (Ahmed, et al. (1994) J. Immunol. Methods 170:211) or by CFSE (carboxyfluorescein diacetate succinimidyl ester) dilution (Tran, et al. (2009) Bioorg. Med. Chem. Lett. 17:225).

CFSE Labeling. CFSE was added to the cell suspensions (1×10⁷ cells/ml) at a final concentration of 5 μM, 37° C., for 30 minutes and the reaction was stopped with fetal calf serum (FCS) at a final concentration of 10%. The cells were washed twice with PBS and resuspended in complete RPMI media.

Flow Cytometry. Indicated populations of T cells were stained (30 minues, 4° C.) with PE-labeled anti-CD25 (Miltenyi Biotec) and FITC-labeled anti-CD127 (eBioscience) antibodies and washed with PBS (containing 0.05% BSA and 0.05% sodium azide). For intracellular staining, cells were fixed and permeabilizied with Fixation/Permeabilization buffer set (00-5523; eBioscience), washed, and stained (30 minutes, 4° C.) with primary antibodies (PE-labeled Foxp3 (PCH101) or PKC-θ (C-18)). Then, the cells were incubated (30 minutes, 4° C.) with FITC-conjugated secondary antibody (Jackson ImmunoResearch Lab. Inc., West Grove, Pa.). Samples were analyzed in a FACSCALIBUR machine (BD, Franklin Lakes, N.J.).

Inhibitors. The PKC-θ inhibitors: compound 20 (C20), BIX02508, BIX02509, BIX02510, and BIX02511 were provided by Boehringer-Ingelheim Pharmaceuticals, Inc (Rigdefield, Conn.) and dissolved in DMSO (Cywin, et al. (2007) Bioorg. Med. Chem. Lett. 17:225; Sims, et al. (2007) Cell 129:773). T cells were pretreated with indicated concentrations of the inhibitors or DMSO control for 1, 5, 15, 30 or 60 minutes at 37° C. and washed three times. The NF-κB (#481408) and IKK (#401474) inhibitors were purchased from CALBIOCHEM (San Diego, Calif.).

RNA Interference. SiRNA duplexes (siRNAs) were synthesized and purified by QIAGEN Inc (Valencia, Calif.) as described (Srivastava, et al. (2004) J. Biol. Chem. 279:29911). The PKC-θ target sequences were: siRNA1 (5′-AAA CCA CCG TGG AGC TCT ACT-3′; SEQ ID NO:1) and siRNA2 (5′-AAG AGC CCG ACC TTC TGT GAA-3′; SEQ ID NO:2); control siRNA was purchased from QIAGEN (1027281). Transfections of freshly purified T cells were performed using the human T cell NUCLEOFECTOR kit (Amaxa Biosystems, Lonza, Base1, Switzerland). Transfected cells were cultured in RPMI 1640 containing 10% FCS on immobilized anti-CD3 antibodies for 48-72 hours. Tranfection efficiency was controlled by evaluating PKC-θ levels using western Blot analysis.

Western Blot. Cells were lysed in RIPA buffer supplemented with protease and phosphatase inhibitors. Equal amounts of protein were loaded on an SDS-PAGE gel and transferred to nitrocellulose membrane. The membranes were blocked, probed with the specific antibodies overnight, washed, and stained with secondary antibodies from LI-COR, Inc. (Lincoln, Nebr.) Immunoreactive protein bands were visualized using an ODYSSEY Infrared Imaging system. Anti-alpha actin antibodies were used as loading controls.

Mice and Mouse Cells. All mice were housed under specific pathogen-free conditions at the Skirball Institute Central Animal Facility, New York University School of Medicine (NYUSM). C57BL/10.PL (Thy1.2), Thy1.1 congenic C57BL/10.PL Thy1.1 and C57BL/10.PL TCR α−/−β^{-/-} mice (Thy1.2) at 6-12 weeks of age from Jackson Laboratory were used. CD4⁺CD25⁺ and CD4⁺CD25⁻ lymphocytes were purified from spleen of wild-type C57BL/10.PL mice by magnetic cell separation using Miltenyi reagents. The purity of the CD25⁺ population was about 90%. CD4⁺CD25⁻CD45RB^{high} fractions were purified by cell sorting in a MOFLO cytometer (BeckmanCoulter) at NYUSM. The purity of MOFLO-sorted fraction was >95%.

Treg Expansion In Vivo. Foxp3⁺ Treg were purified from Foxp3-GFP knockin mice (Bettelli, et al. (2006) Nature 441:235) by cell sorting in a MOFLO cytometer (BeckmanCoulter) at NYUSM. Sorted cells were treated with C20 for 30 minutes at 1 μM and washed. C57BL/10.PL TCR α^{-/-}β^{-/-} mice were intravenously injected with 1.5×10⁶ sorted CD4⁺CD25⁻CD45RB^{high} T cells in combination with 5×10⁵ of Foxp3-GFP Treg. Numbers of Foxp3-GFP Treg were evaluated in spleen and mesenteric lymph nodes after 7 days by flow cytometry.

T Cell Transfer Model of Colitis. For T cell transfer model of colitis, C57BL/10.PL TCR α^{-/-}β^{-/-} mice were intravenously injected with 5×10⁵ sorted CD4⁺CD25⁻CD45RB^{high} T cells alone or in combination with 0.125×10⁵ of CD4⁺CD25⁺ T cells that were pretreated or not as indicated. Mice were weighed twice a week and inspected for clinical signs of colitis such as diarrhea, rectal prolapse and ruffled fur. Scoring was carried out by histological examination, according to known methods (Powrie, et al. (1996) J. Exp. Med. 183:2669; Ding, et al. (2008) Nat. Med. 14:162). Paraffin-embedded sections were histochemically stained with Hematoxylin and Eosin. Images were acquired with an AXIOPLAN 2 fluorescent microscope (ZEISS). P values were determined by Mann-Whitney test or two-tailed t-test by using the GRAPHPAD PRISM software (San Diego, Calif.)

### Example 2

### Protein Kinase C-θ Mediates Negative Feedback on Regulatory T Cell Function

To study signaling in human Treg IS, a model system was developed on supported planar bilayers containing the mobile fluorescently labeled adhesion molecule ICAM-1 and antigen surrogate anti-CD3 (the signaling subunit of the TCR) antibodies, using CD4⁺CD25⁺ Teff or CD4⁺CD25⁺ Treg freshly isolated from peripheral blood. Teff and Treg both formed IS, defined by a symmetric pattern composed of a central cluster of anti-CD3 surrounded by a ring of ICAM-1 (Grakoui, et al. (1999) Science 285:221; Monks, et al. (1998) Nature 395:82). Treg IS were more stable than Teff IS, which displayed symmetry breaking within 20 minutes (Sims, et al. (2007) Cell 129:773). Ex vivo expanded human umbilical cord blood Treg (Godfrey, et al. (2005) Blood 105:750) displayed similar behavior to adult peripheral blood Treg. Recruitment of TCR proximal signaling molecules to IS were measured by staining with phospho-Src kinase activation loop and ZAP-70 kinase interdomain A tyrosine 319 antibodies and imaging with total internal reflection fluorescence microscopy (TIRFM) (Campi, et al. (2005) J. Exp. Med. 202:1031). Signals were quantified based on unbiased measurement of IS proximal fluorescence intensity. Teff IS displayed significantly higher amounts of phospho-Src than Treg; however, a similar intensity of phosphorylation of the downstream kinase ZAP-70 was observed. Because ZAP-70 phosphorylation appeared normal in Treg, the protein kinase C-θ (PKC-θ) pathway, which is downstream of Src family kinases (Hayashi & Altman (2007) Pharmacol. Res. 55:537) and mediates IS breaking (Sims, et al. (2007) supra), was explored.

PKC-θ recruitment to the IS leads to recruitment of Carma-1, a MAGUK (membrane-associated guanylate kinase) protein, that enables the assembly of a Carma-1-Bc110-Malt1 complex necessary for NF-κB activation and subsequent Teff activation (Rawlings, et al. (2006) Nat. Rev. Immunol. 6:799). PKC-θ and Carma-1 recruitment in IS were quantified on planar bilayers by TIRFM. Teff IS recruited PKC-θ in a broad pattern overlapping with early TCR signaling, as previously reported (Sims, et al. (2007) supra). Treg displayed six-fold lower PKC-θ recruitment to IS than Teff and this recruitment was focused in a limited area defined by a small number of bright puncta. The same difference in the ability to recruit PKC-θ was found with ex vivo expanded human umbilical cord blood Treg and expanded CD4⁺ CD25⁻ Teff cells. CD28 co-stimulation plays an important role in PKC-θ recruitment to IS (Monks, et al. (1998) supra; Hayashi & Altman (2007) supra). Thus, the ability of Treg and Teff to recruit PKC-θ in the presence of a CD28 ligand, CD80, were compared in the bilayer. The co-stimulatory signal increased the amounts of PKC-θ recruited to IS in both Treg and Teff. Even in the presence of CD80 in the bilayer, the Treg still have significantly less PKC-θ recruited to the IS than Teff (P<0.001). Interestingly, total PKC-θ expression was even higher in Treg compared to Teff. Moreover, the confocal imaging revealed that PKC-θ was sequestered away from the Treg IS, whereas PKC-θ in Teff accessed the IS and formed cytoplasmic puncta. Treg IS also displayed significantly less Carma-1 recruitment than Teff IS. Thus, recruitment of PKC-θ and its down-stream target to the IS are reduced in Treg.

The TCR and LFA-1 integrin-dependent components of PKC-θ recruitment were further dissected using bilayers containing anti-CD3 or ICAM-1 only, respectively. TCR engagement in Teff was necessary and sufficient for increased PKC-θ recruitment based on a two-fold increase in PKC-θ fluorescence intensity on anti-CD3 only bilayers compared to ICAM-1 alone bilayers. TCR engagement alone in Treg recruited 11-fold less PKC-θ than in Teff, whereas LFA-1 engagement alone recruited 3.5-fold more PKC-θ than in Teff. Thus, TCR triggering in Treg actually down-regulates PKC-θ recruitment to the IS by 7.7-fold compared to basal recruitment by LFA-1 engagement alone.

It was posited that PKC-θ activation may be part of a negative feedback loop controlling Treg function because TCR signals are essential for Treg function, but suppress PKC-θ recruitment. Accordingly, it was determined whether inhibition of PKC-θ may affect the suppressive function of human CD4⁺CD25⁺ Treg cells. Teff function was measured as cytokine secretion and cell proliferation. C20 treatment of only the Tregs significantly up-regulated their suppressive ability, even in the presence of CD28-mediated costimulation, but did not induce suppressive activity in treated Teff. Consistent with imaging data showing that co-stimulation up-regulates the recruitment of PKC-θ to IS in Treg, untreated Treg demonstrated reduced ability to suppress Teff function in the presence of CD28 antibodies (40% and 25% respectively), and this difference was abrogated by treatment with C20. Inhibition of PKC-θ seemed to increase Treg suppressive function in general, without preference for specific helper T cell type. C20 also significantly increased Treg function in an antigen presenting cell-dependent assay. It has been reported that the CD4⁺CD25^{high} Tregs are the most potent suppressors in vitro (Shevach (2009) Immunity 30:636); therefore CD4+ T cells were sorted according to their CD25 expression, the sorted Treg were pretreated with C20 and the CD4⁺CD25^{int} or CD4⁺CD25^{high} T cells were co-cultured with target CD4⁺CD25⁻ T cells. Although the CD4⁺CD25^{high} T cells manifested a greater suppressive activity, pretreatment with C20 significantly enhanced their suppression of IFN-γ secretion. Finally, the effect of C20 on Treg function was time-dependent; the peak of suppressive function was observed after 30 minutes of treatment. TCR-induced DNA binding of NF-κB p65 and p50 subunits, which indicates NF-κB activation, and the ability to proliferate were greatly reduced by C20 in both Treg and Teff.

To test whether activation of NF-κB was a critical PKC-θ target in control of Treg activity, NF-κB activation was inhibited using two different types of inhibitors, NF-κB and IKK inhibitors. This analysis indicated that both inhibitors significantly increased Treg activity. Moreover, treatment of Treg with analogs of C20 with different IC₅₀ values demonstrated that of the analogs tested, only PKC-θ inhibitors with IC₅₀≦1 nM significantly upregulated Treg suppressive function. Finally, neutralizing antibodies against TGF-β receptor II completely blocked the suppressive function of Treg induced by inhibition of PKC-θ either in APC-free or in presence of APC in the co-culture, indicating the possible involvement of TGF-β presented by Treg as a suppressive mechanism.

To confirm the conclusion that inhibition of PKC-θ up-regulates the suppressive activity of human Treg, PKC-θ gene expression was specifically silenced using RNA interference (Srivastava, et al. (2004) J. Biol. Chem. 279:29911). The specific PKC-θ siRNA reduced PKC-θ expression by 80%. Moreover, silencing of PKC-θ significantly increased Treg-mediated suppression of IFN-γ secretion by Teff. PKC-θ silencing in Teff resulted in the expected down-regulation of IFN-γ secretion and cell proliferation. In summary, it was concluded that PKC-θ activity induced by TCR signaling mediates a negative feedback loop that reduces the activity of human CD4⁺CD25^{high} Treg to suppress cytokine secretion and proliferation of Teff in vitro.

Rheumatoid arthritis (RA) is a chronic autoimmune disorder that results in the destruction of joint architecture (Feldmann (2002) Nat. Rev. Immunol. 2:364). Recent studies in RA patients demonstrated that the function of CD4⁺CD25^{high} Treg is impaired (Valencia, et al. (2006) supra; Ehrenstein, et al. (2004) supra). Treg purified from peripheral blood of 25 RA patients with different severities of disease were used in the analysis herein. This analysis indicated that despite the anergic state and comparable Treg numbers to healthy donors, RA Treg demonstrated significantly reduced suppression of IFN-γ from autologous CD4⁺CD25⁻ Teff. The loss of function was due to defective intrinsic function of Treg from RA patients and not due to increased resistance of Teff (Valencia, et al. (2006) supra). Treatment with C20 significantly increased the suppressive function of Treg purified from all 25 RA patients to levels comparable with healthy donor-derived Treg (30-50% inhibition at a Treg/Teff of 1:3). Moreover, the defective Treg function in RA patients was inversely correlated with the Disease Active Score (DAS score) and the shift in IFN-γ secretion was similar across the disease score spectrum. Thus, inhibition of PKC-θ boosts the suppressive function of Treg isolated from RA patients independent of the severity of disease.

Treg treatment with TNF-α inhibits their activity and down-regulates expression of the Treg master regulator transcription factor FoxP3 (Valencia, et al. (2006) supra). The possibility that elimination of the PKC-θ mediated negative feedback on Treg function may render Treg resistant to inhibition by TNF-α was subsequently investigated. In the presence of TNF-α, Treg displayed significantly reduced inhibition of IFN-γ secretion and proliferation in Teff, and this effect was largely reversed by C20 or PKC-θ-specific siRNA. Moreover, C20 prevented TNF-α-induced down-regulation of Foxp3 in Treg. Strikingly, TNF-α treatment induced increased PKC-θ recruitment to IS in Treg by decreasing sequestration at the distal pole, consistent with the idea that TCR activated PKC-θ mediates negative feedback on Treg function that is further enhanced by TNF-α.

Subsequently, the ability of C20 to increase Treg function in vivo was determined using a colitis model in TCR α^{-/-}β^{-/-} mice induced by transfer of the CD4⁺CD25⁻CD45RB^{high} Teff cells (Powrie, et al. (1996) J. Exp. Med. 183:2669; Ding, et al. (2008) Nat. Med. 14:162). Treatment of murine Treg with C20 up-regulated their suppressive function in vitro. Moreover, C20-treated CD4⁺CD25⁺ Treg cells provided significant protection from colitis, as demonstrated by normal weight gain and normal histology of the distal colon in 7 of 8 mice. C20 treatment increased the number of Treg recovered from mesenteric lymph nodes and the spleen. This protection was significantly greater than afforded by Treg left untreated. Treatment of CD4⁺CD25⁻ Teff with C20 prior to transfer with untreated Teff did not protect mice from colitis. Thus, PKC-θ inhibition significantly increased the suppressive effect of Treg in vivo.

Treg function is crucial to prevent autoimmunity in mice and humans. Although Treg numbers in, patients suffering from autoimmune diseases are similar to healthy controls, Treg function is defective, probably due to negative regulation of Treg by the inflammatory milieu (Shevach (2009) supra). In the present study it was demonstrated that PKC-θ is sequestered in the distal pole of Treg cells in a manner that reduces its recruitment to the IS. Moreover, inhibition of PKC-θ protects both mouse and human Treg against negative effects of TNF-α, which appears to act in Treg by unleashing PKC-θ from the distal pole. In Teff, PKC-θ is part of a strong positive circuit with free access to TCR signalosomes in the IS, NF-κB activation, increased expression of IL-2, proliferation and survival (Hayashi & Altman (2007) supra; Monks, et al. (1997) Nature 385:83; Sun, et al. (2000) Nature 404:402). PKC-θ is not sequestered in the distal pole of Teff, although the negative regulators like Csk binding protein are (Shaffer, et al. (2009) J. Immunol. 182:1021). In contrast, it was noted that Treg display increased IS stability, consistent with decreased PKC-θ activity in the IS and attenuated NF-κB activation. The proposed role of NF-κB in inhibition of Treg function is consistent with the system wide role of this family of transcription factors in promoting inflammation (Barnes & Karin (1997) N. Engl. J. Med. 336:1066). IS stabilization may enhance Treg function based on recent evidence for an important role of IS in Treg effects mediated through DC (Sakaguchi, et al. (2008) supra; Sarris, et al. (2008) Immunity 28:402).

It has now been demonstrated that formation of IS induces altered signaling pathways in Treg, characterized by reduced recruitment of Src kinases, PKC-θ and Carma-1 to the IS. Moreover, in Treg, PKC-θ acts as a pro-inflammatory mediator and this effect is enhanced by TNF-α. Indeed, Treg treated with C20 displayed enhanced ability to prevent autoimmune colitis and restore function of Treg from RA patients. Thus, targeting the PKC-θ-mediated negative feedback loop enhances the activity of Treg and makes them resistant to cytokines associated with the inflammatory milieu found in some autoimmune diseases. Thus, inhibition of PKC-θ in Treg finds use as a valuable component in Treg adoptive immunotherapy to treat autoimmunity and graft versus host disease (Riley, et al. (2009) Immunity 30:656).

What is claimed is:

1. A method for reducing tumor necrosis factor activation of regulatory T cells comprising contacting a regulatory T cell with a Protein Kinase C theta (PKC-θ) inhibitor in the presence of tumor necrosis factor so that activation of the regulatory T cells is reduced.

2. The method of claim 1, wherein the PKC-θ inhibitor is a protein-based, oligonucleotides-based or small organic molecule inhibitor.

3. A method for restoring the activity of a defective regulatory T cell comprising contacting a defective regulatory T cell with a Protein Kinase C theta (PKC-θ) inhibitor so that the activity of the regulatory T cell is restored.

4. The method of claim 3, wherein the PKC-θ inhibitor is a protein-based, oligonucleotides-based or small organic molecule inhibitor.

5. The method of claim 3, wherein the defective regulatory T cell is from a rheumatoid arthritis patient.

6. A method for enhancing the function of a regulatory T cell comprising contacting a regulatory T cell with a Protein Kinase C theta (PKC-θ) inhibitor so that the function of the regulatory T cell is enhanced.

7. The method of claim 6, wherein the PKC-θ inhibitor is a protein-based, oligonucleotides-based or small organic molecule inhibitor.

8. A method for facilitating adoptive immunotherapy, comprising contacting isolated regulatory T cells with a Protein Kinase C theta (PKC-θ) inhibitor and administering said regulatory T cells to a subject in need thereof thereby facilitating adoptive immunotherapy of the subject.

9. The method of claim 8, wherein said administration prevents autoimmune colitis.

10. The method of claim 8, wherein the subject has rheumatoid arthritis.

11. The method of claim 8, wherein the subject has graft rejection or graft-versus-host disease.

12. The method of claim 8, wherein the PKC-θ inhibitor is a protein-based, oligonucleotides-based or small organic molecule inhibitor.


SEQUENCE LISTING (3 sequences)

SEQ ID NO: 1 - DNA, 21 bp, Artificial sequence
  Synthetic oligonucleotide
aaaccaccgtggagctctact

SEQ ID NO: 2 - DNA, 21 bp, Artificial sequence
  Synthetic oligonucleotide
aagagcccgaccttctgtgaa

SEQ ID NO: 3 - protein, 18 aa, Artificial sequence
  Synthetic peptide
LHQRRGAIKQAKVHHVKC